(19) United States
(12) Patent Application Publication
(10) Pub. No.: US 2024/0209320 A1
(43) Pub. Date: Jun. 27, 2024

(54) SYSTEM AND METHOD FOR ISOLATING, STORING AND USING CELLS

(71) Applicant: THE MEDICAL RESEARCH, INFRASTRUCTURE, AND HEALTH SERVICES FUND OF THE TEL AVIV MEDICAL CENTER, Tel Aviv (IL)

(72) Inventors: Nir SHANI, Ramat Hasharon (IL); Inna SOLODEEV, Ramat Gan (IL); Adiel SHFARAM, Herzliya (IL)

(21) Appl. No.: 18/574,249

(22) PCT Filed: Jun. 21, 2022
(86) PCT No.: PCT/IL2022/050663

§ 371 Date: Dec. 26, 2023

Related U.S. Application Data
(60) Provisional application No. 63/202,686, filed on Jun. 21, 2021.

(51) Int. Cl. C12N 5/0775; C12M 1/00; C12M 1/26; C12M 1/33; C12M 3/00

(52) U.S. Cl. CPC C12N 5/0667; C12M 23/40; C12M 23/42; C12M 33/22; C12M 45/02; C12M 45/09; C12M 47/04; C12N 2509/00

(57) ABSTRACT

The invention generally provides a device and a method for isolating cells from tissues and using same in vivo or in vitro.

### TECHNOLOGICAL FIELD

The invention generally contemplates systems and methods for isolating cell populations from a variety of tissues.

### BACKGROUND OF THE INVENTION

Cultured mesenchymal cells were termed mesenchymal stem cells (MSCs) to denote their multipotent differentiation potential. The gold standard for isolation of stromal cells from fat includes the enzymatic dissociation of cells by collagenase followed by centrifugation which separates a stromal vascular fraction (SVF) precipitate from the adipocyte tissues which remain at the upper fluid layer, due to their high fat content. The SVF separates as a pellet which may be isolated. Following their isolation, SVF cells can be utilized for clinical purposes either fresh, immediately following their isolation, or as MSCs following their tissue culture expansion.

Traditionally, SVF isolation involved the manual introduction of fat tissue into a device containing an enzyme or a mechanical means for disrupting the cells and subsequent manual removal of the cells pellet from the device. Storage for later use have been limited as storage required further manipulation and transfer of the cells into a further container suitable for cryogenic storage. A major impediment to storage and later use of SVF cells or to a larger scale production of SVF cells has been the lack of a device and a method to effectively and efficiently isolate, preserve and store SVF cells, as well as other similarly produced cells in a highly sterile form that does not introduce any sources of contamination and which can be carried from sample extraction directly from a human body and cells return directly to a human body in a single methodological clinically acceptable step.

The nature of living cells produced by means of enzymatic or mechanical technologies makes development of highly sterile and long-term stored cells samples challenging. Publication [1] attempts to provide a solution to at least part of the problem by providing a system for enriching a cell population from a mammalian adipose tissue which utilizes a device that is configured to receive the fat tissue directly from the subject and manipulating the tissue within a single sterile container. The container is adapted to receive, manipulate and permit separation of the enriched cell population. It} cap configured to allow the attachment of an electrical actuator; a mechanical tissue disruption device detachably connected to the electrical actuator and situated within the container assembly being adjacent to but having no contact with base of the container; and a vacuum pump to activate liposuction of adipose tissue into the container via an aspiration tube through an inlet in the closing cap of the container assembly. This system, however, requires isolation of the enriched fraction from its sterile environment, possibly into a different container which may be used for storage.

### BACKGROUND PUBLICATIONS

[1] WO 2016/199149

### GENERAL DESCRIPTION

The emerging fields of tissue engineering, tissue regeneration and immune regulation are poised to make enormous progress in a vast gamut of clinical protocols, in both the research and clinical arenas. Use of isolated cell fractions, in general, or specifically SVF fractions, thus requires the immediate availability of such fractions in viable and sterile forms, in a clinical setting that produces the cells, manipulates same to isolate an enriched fraction and use the cells or store them while minimizing sources of contamination which can risk cell viability and future use. Accordingly, what is needed in the art are improved methods and systems for isolation, processing and storage of cells under conditions that are routinely required at the point of use.

The inventors of the technology disclosed herein have developed a unique device and methodology for isolating or separating cells or cell populations from tissues and enabling their further use and storage without needing to resort to using multiple containers or steps. In most general terms, the inventors provide a device having a plurality of detachable parts which can be assembled into a single device and operated to obtain a tissue sample through a first opening thereof and deliver an isolated cell fraction from another opening directly to a subject's body, such that all processing steps from tissue to enriched cell population are achievable in a single closed and sterile container assembly. The device may be additionally configured to hold and contain the cell fraction under conditions that permit long term storage, e.g., cryogenic storage, in a ready-for-future-use condition.

The novel device of the invention provides for the first time a high that can accommodate all processing steps: cells isolation, cells enrichment, cell fraction collection, cell fraction isolation, cell fraction storage and cell fraction clinical utilization, while maintaining sterility, viability and functionality of the cells.

More specifically, a device of the invention comprises two or more detachable parts which may be assembled and detached during use to improve isolation of cells and further enable their storage or immediate use under highly sterile conditions. Whether used for clinical purposes immediately following their isolation, or as mesenchymal stem cells (MSCs) following tissue culture expansion, the isolated cells do not require manipulation or further handling before use. The device may comprise a plurality of parts, two of which being (i) a cell isolation unit and (ii) a cell delivery unit. Cells isolated in one unit of the device are gravitationally collected in the other unit of the device, which is then detached and may be used as a delivery unit, as further explained herein. Various configurations of devices of the invention may also comprise cell filtration units, medium reservoirs, analytical sampling modules and others.

Thus, in a first aspect of the technology, there is provided a device comprising a cell isolation chamber (or unit), herein referred to as CIC, detachably associated with a cell delivery unit, the CIC is adapted to receive a tissue to be mechanically and/or enzymatically manipulated to separate therefrom cells or cell populations, wherein the device is configured to allow cells isolated from the tissue in the cell isolation chamber to gravitationally collect in the detachable cell delivery unit, wherein the delivery unit is configured for delivery of the isolated cells in vivo or in vitro.

The invention further provides a device comprising:
a cell isolation chamber or unit (CIC) configured for separating/isolating cells or a cell population from tissues containing same, said CIC being adapted to hold a tissue and a medium optionally containing an enzyme;
optionally a tissue disruption element for mixing or mechanically disrupting the tissue to obtain the cells or the cell population (wherein said tissue disruption element may or may not be present when the container unit contains an enzyme);
a delivery unit being detachably connected to the CIC through a connector associated to a valve assembly for affecting material communication with said CIC for receiving thereinto an amount of the CIC content (e.g., separated/isolated cells or cell population), e.g., wherein optionally the CIC content (separated/isolated population) is in a form of a pellet;
wherein the delivery unit having a main body (e.g., a barrel) and a reciprocating unit or a member or a pump configured for expelling its content (i.e., said separated/isolated cells or cell population or a pellet of same).

Also provided is a cell isolation chamber (CIC) for separating/isolating cells or a cell population from tissues containing same, said CIC being adapted to hold a tissue and a medium optionally containing an enzyme; wherein said CIC comprising optionally a tissue disruption element for mixing or mechanically disrupting the tissues to obtain the cells or the cell population (wherein said tissue disruption element may or may not be present when the CIC contains an enzyme); and wherein said CIC is configured to detachably associate or connect to a delivery unit through a valve assembly permitting material communication between the CIC and the delivery unit, said delivery unit being configured for receiving at least a part of a content from said CIC (e.g., separated/isolated cells or cell population or medium), wherein optionally the content (i.e., separated/isolated cells or cell population) is in a form of a solid or a pellet; wherein the delivery unit having a main body (e.g., a barrel) and a unit or member such as a reciprocating pump, for expelling said content therefrom.

The CIC may be of any shape and volume and may be adapted for isolation of cells from tissues. As the CIC is configured and operable to treat large volumes of fat tissue, the CIC is typically not a test tube or any other experimental small-volume or small-sized container. Typically, the CIC is conically shaped or has a conical interior or a conically shaped inner base, wherein the detachable delivery unit is connected or associated, directly or indirectly, to the end of the conical interior end, e.g., to the tip of the conical interior. The CIC is additionally configured or adapted to received or be equipped with a disruption element suitable for tissue disruption, which maintains cell viability. Such elements may be known in the art, namely a rotatable blade and/or set of blades rotating in the same or opposite directions, a vibrating rotor, a grinding unit which comprises or consists balls or beads or any other crushing elements. Where the element is operated, e.g., the rotor may be connected to an actuator.

In some embodiments, the CIC may be provided with an element or a member configured for inducing vortexing, shaking or strong stirring, wherein the member is not a blade or a set of blades.

In some embodiments, the CIC may be provided with a rotating element or a member that is not a blade.

In some configurations, the CIC is configured or adapted to receive or hold a mechanical tissue disruption unit that is controlled by an electrical actuator being external and detachably connected to the CIC, optionally through a closing cap of the CIC. The mechanical tissue disruption unit may be in a form of a rotor or a blade or a member selected to affect a rotational force. The disruption unit may be configured to be lowered and retracted to and from the concave bottom of the CIC and to be locked in place during tissue processing. The rotor speed (RPM) may be adapted to maintain cells viability, preserving cell integrity and preventing foaming and protein denaturation. In some embodiments, the rotor may be selected to rotate at between about 500 RPM and about 8,000 RPM, or between about 1,000 and 6,000 RPM, or between about 1,100 and 1,500 RPM or between 1,200-2,200 RPM.

In addition to the tissues, the CIC is further adapted to receive a liquid or a medium which may or may not comprise an enzyme or chemicals assisting in dissociating or digesting the tissues to release the cells. The liquid or medium may be received via one or more inlet members, typically positioned at the top part of the CIC unit.

The one or more inlet members may be shaped as one or more openings for introducing into the CIC, e.g., by suction, or by any others means, a tissue sample from which cells are to be separated. The inlet member(s) may be each equipped with a tubing or an elongated conduit permitting association to an aspiration unit, to a filtration unit, to a fat collection unit or to any other fat or tissue processing unit, or directly to a region of a subject's tissue. An aspiration tube may be used for collecting tissue directly from a subject's body or from any holding container such as those used to receive fat tissue during liposuction procedure. The inlet element(s) may be in a form of an opening allowing introduction of a tissue sample obtained separately, at a time period prior to isolation of the cells.

In cases where the inlet member, one or more, is equipped or adapted with an aspiration tube, the aspiration tube may be a cannula having a hollow tube blunted at the tip and having at least one opening (holes) at its tip. The cannula may be reusable (to allow multiple uses thereof). The cannula's hollow tube may be configured to allow passage of a mixture comprising liquid and fat tissue components suctioned from a subject's body into the CIC through an inlet closing cap which may be provid. with the cannula or with the CIC.

Alternatively or additionally, one or more or all of the inlet members may be provided with closing caps adaptable to be removed or to be punctured or penetrated to receive therethrough a tubing that may be connected to any auxiliary device or container to allow for sterile and effective delivery of a tissue sample or liquid media or any other solid or liquid component into the CIC.

The inlet members as any potentially present outlet members provided on the body of the CIC may be adapted with a valve member. These valves, different from the valve assembly provided in a connector associating the CIC and the delivery unit, as further described herein, may be configured for association to a tubing or a material conduit of any shape and form.

Isolated cells may settle to the bottom of the CIC without further treatment or maybe forced to collect and settle via use of a centrifuge or vacuum. Subsequently, the cells may gravitationally collect in the delivery unit, e.g., with or without assistance of vacuum or a pressure gradient. In the CIC or after their transition into the delivery unit, the cells or pellet of cells may be washed. To provide efficient washing, the CIC may be provided with a detachable or a fixed mixing member actuated to stir the medium contained in the CIC. The mixing member may be a mechanical disrupting unit described herein or any other member permitting mixing of the CIC content. Such mixing member may operate as an axial flow impeller or as a radial flow impeller.

Once the cells or pellet or any part of the CIC content, including, e.g., medium, fat, unprocessed tissues, fibers, etc, collect in the delivery unit, the delivery unit containing the cells or pellet of cells may be disassociated or detached from the CIC and used to deliver the cells or pellet of cells based on the intended use, or stored for any time period, under any storage conditions in the delivery unit, which may be subsequently used to deliver the content, e.g., cells or pellet of cells or tissues, without needing to resort to post treatment or manual handling.

Cells isolated in the CIC may be filtered to be separated from other components of the tissue or medium or may be enriched by size before entering the delivery unit or following storage in the delivery unit, prior to use. Filtration or separation between cell populations may be achievable by any filtration or separation or enrichment means suitable in the field.

In some embodiments, the cells or pellet or tissue are/is further pr specific desirable cell population, e.g., using fluorescence-activated cell sorting (FACS), gel gravitation separation, column-based techniques or any other suitable cell enrichment method known to the skilled artesian.

In some embodiments, the cells or pellet of cells may be used clinically for, e.g., immune regulation for treating or preventing autoimmune diseases, immune disorders, such as graft versus host disease, COVID-19, acute respiratory distress syndrome, and others; tissue regeneration, e.g., fat grafting, hair growth, bone regeneration, nerve regeneration, skin rejuvenation, wound healing and others; for the treatment or prevention of stroke, perianal fistulas, rheumatic disease, general arthritis, osteoarthritis and others.

The delivery unit may be shaped and operated as a storage unit or as a syringe enabling e.g., systemic delivery of the cells, for example, via intravenous or intra-arterial, intramuscular or intraperitoneal delivery; or locally by intra-articular delivery into the spine, into the scalp (hair growth), into the face/breast with or without a fat graft, in a scaffold (bone regeneration, breast degradable scaffolds) or by other means. In other words, the delivery unit may be used as a delivery tool for medicinal or therapeutic or cosmetic or aesthetic purposes. The delivery unit may similarly be used for research purposes.

To enable variable use, the delivery unit in a device of the invention may have a main body shaped as a barrel or an elongated tubular structure comprising a unit, such as a reciprocating pump, or a plunger and a plunger rod (a syringe) that is configured for linear movement within the barrel or elongated tubular structure. The barrel may have at least two openings: one of which positioned along a path of material communication with said CIC and another at one end of the barrel or structure being opposite to the plunger path and through which the cells may be expelled from the delivery unit. Thus, the delivery unit may be shaped and operable as a syringe having an opening for association with the CIC. The opening may be adapted with a valve assembly or a fitting enabling direct attachment to and detachment from the CIC. The opening may be positioned mid-way along the barrel length or at any distance from the barrel opening. Typically, the opening enables perpendicular attachment of the container unit to the delivery unit.

The delivery unit may be configured as an elongated tubular structure comprising a reciprocating pump unit or a plunger and a plunger rod (a syringe) that is configured for linear movement within the barrel or elongated tubular structure. The plunger rod may be of a length ending the full or substantially full length of the inner cavity of 1 such that its end contacts the opposite end of the barrel. Alternatively, the plunger rod may extend only a part of the structure inner length.

In some embodiments, the tubular structure is provided with a membrane or a filter disposed perpendicularly to a main axis (or flow axis—the direction of material flow) of the structure and configured to selectively discharge from the structure materials that cross the membrane or filter and/or collect particulate matter or fibers of larger dimensions which do not cross the membrane or filter and collect thereon.

The delivery unit may be connected with the CIC or the main body of the device or tip end of the conical container, as described herein, through any connector member or fittings known in the art. To securely assemble the CIC and the delivery unit, each is provided with a connector member which may be a male or female member having a threaded or non-threaded end(s); a male or female barb connector; a coupling adaptor member; a double tapped bushing adaptor; and others.

The one or both connector members may be associated to a valve member to form a valve assembly such that once both the CIC and the delivery unit are securely assembled via a connector member provided on each respectively, as defined, a valve assembly may be operated to controllably permit liquid communication from the CIC to the delivery unit. The valve assembly may comprise one or more valve members, which together are designed to ensure liquid communication from the CIC to the delivery unit upon demand and in a controllable manner.

In some embodiments, a valve member may be a one way-flap member that allows flow in a direction from the CIC to the delivery unit and stops flow in the direction of origin.

In some embodiments, a valve member may be an electric valve operated by a motor or a mechanical valve member operated manually.

In some embodiments, one or more of the valve members is a manual valve. Manual valve members may be in a form selected from levers, pedals, hand knobs or wheels to crank a screw-turned stem.

In some embodiments, the valve member may be a Luer valve.

In some embodiments, the valve assembly is or comprises a single valve member that is provided in the connector member of the CIC unit or in the connector member of the delivery unit.

In some embodiments, the valve assembly is or comprises a double two valve members, one associated with the CIC unit and another with the delivery unit.

In a double valve unit, the two valves define an upper valve body (associated with the CIC) and a lower valve body (associated with the delivery unit), which may have the same structure, and may be coaxially formed. The two ends of the upper valve body and the two ends of the lower valve body are integrally connected to their respective units and to the two connector members.

In some embodiments, each valve member in a double valve unit comprises a body, an inlet end and an outlet end. The inlet end of the CIC valve is connected to the body of the CIC, and the outlet end defines a connector member capable of associating to a connector end of the delivery unit, which defines an inlet end of the delivery unit. The outlet end of the delivery unit valve is connected to the body of the delivery unit.

The valve assembly or one or each of the valve members may be adapted with an optical sensor that operates the valve to control material flow into the delivery unit. for example, the sensor, through the use of a controller, may set flow of material into the delivery unit to a specific or predetermined volume threshold. The sensor may alternatively report passing of cells or cell pellet and prevent further flow of material.

The device of the invention may be assembled such that the CIC unit is above the delivery unit to enable gravitational collection of the CIC content, e.g., cell pellet in the delivery unit, without external intervention. In some embodiments, a part of the CIC content may be communicated to the delivery unit via use of a pump or an evacuated delivery unit. The CIC may be equipped with a base or a stage or a leg assembly which maintains the CIC at a distance from the surface on which it is positioned sufficient to fit the delivery unit at its lower part. Thus, in some embodiments, the body of the device or CIC unit is supported on legs which may be adjustable to a distance convenient for operation.

Devices of the invention provide for continuous manipulation of a tissue sample to maximize yield of a cell population. While a method for isolating a desired cell population may be carried out utilizing an enzymatic manipulation or a mechanical manipulation of the tissue sample to produce a cell population of a given composition and optimal yield, the device also enables multiple tissue manipulation steps which involve both enzymatic and mechanical manipulation of the same tissue sample. This maximizes cell population isolation.

Thus, a method for isolating a cell population from a tissue utilizing the invention may involve conditions enabling enzymatic treatment of the tissue or conditions enabling mechanical treatment of the tissue; or consecutive steps of mechanical treatment followed by enzymatic treatment, providing isolation of two separate enriched fractions which combined yield is greater than a yield expected from each tissue manipulation step alone. Similarly, a method may involve steps of repeated cell isolation using same conditions, e.g., mechanical or enzymatic, wherein the tissue initially treated is continuously treated to separate more of the desired cell population, such that each fraction isolated is delivered to a different delivery unit.

The invention thus provides a method for isolating a cell population from a tissue, the method comprising:
disrupting or digesting the tissue in a device comprising a cell isolation chamber (CIC) and a delivery unit detachably associated with the CIC, using a tissue disruption unit or an enzymatic solution present in CIC to obtain a cell suspension; and
permitting the suspension or a part of the CIC content to settle into the delivery unit through a valve assembly, wherein the delivery unit is configured for in vivo or in vitro use, as disclosed herein.

In some embodiments, different fractions of the isolated cells obtained from the same tissue contained in the CIC may be obtained by repeating the disrupting or digesting step one or more times on the same tissue contained in the CIC to further separate additional fractions of cells. Each of the fractions may be delivered into a different delivery unit, whereby between each repeated step, a delivery unit contacting a previous fraction is replaced with a further delivery unit.

The invention also provides a method for isolating different fractions of a cell population from a tissue, the method comprising:
disrupting or digesting the tissue in a device comprising a cell isolation chamber (CIC) and a delivery unit detachably associated with the CIC, using a tissue disruption unit or an enzymatic solution present in CIC to obtain a cell suspension; and
permitting the suspension or a part of the CIC content to settle into the delivery unit through a valve assembly.
wherein the disrupting or digesting is repeated one or more times on the tissue contained in the CIC to further separate additional fractions of cells into different delivery units.

Also provided is a method for isolating a cell population from a tissue, the method may comprise:
disrupting or digesting a tissue in a device comprising a cell isolation chamber (CIC) and a delivery unit detachably associated with the CIC, using a tissue disruption unit or an enzymatic solution present in CIC to obtain a cell suspension;
optionally washing a suspension of cells within the CIC, optionally when the container unit is attached to the delivery unit;
optionally transferring the device comprising an assembled CIC/delivery unit or the CIC unit only into a centrifuge and centrifuging same to thereby isolate cells or a cell pellet;
and
permitting the cells or cell pellet to settle into the delivery unit through a valve assembly permitting material communication from said CIC.

In some embodiments, the tissue is treated, i.e., under enzymatic or mechanical conditions, when the delivery unit is detached from the CIC; wherein prior to permitting the cells or cell pellet to settle into the delivery unit, the delivery unit is attached to the CIC and the valve assembly is positioned to provide material flow into the delivery unit.

In some embodiments, the cells or cell pellet or otherwise at least a part of the CIC content are/is gravitationally collected in the delivery unit.

In some embodiments, an amount of the CIC content is permitted to gravitationally collect in the delivery unit, said content comprises or consists the cells or cell pellet.

In some embodiments, the cells or cell pellet or CIC content is collected in a liquid medium. The volume of the medium collected in the delivery unit may be varied based on the volume of the delivery unit and the intended use.

In some embodiments, the cells or cell population are collected in one delivery unit which is detached and replaced with another delivery unit to receive another fraction of the cells or to receive another portion of the CIC content, e.g., in a form of a liquid medium.

As discussed herein, material flow or communication from the CIC to the delivery unit is enabled through a valve assembly, e.g., a double valve, which typically separates the CIC from the delivery unit and prevents uncontrolled or unwanted or untimed flow of material or content from the CIC into the delivery unit. When material tran delivery unit is to be prevented, e.g., during processing of the tissue, or after the cells or cell pellet has been collected in the delivery unit, the valve member or valve assembly is operated automatically, electrically or manually into a ‘close’ configuration. The ‘close’ configuration may be achieved by closing one or all valve members provided in the connector assembly. When material flow of content from the CIC to the delivery unit is to be permitted, the valve member or valve assembly is operated automatically, electrically or manually into an ‘open’ configuration. The ‘open configuration requires all valve members to be at the open configuration to permit flow.

Thus, in some embodiments, any of the methods disclosed herein may comprise a step of operating a valve member or a valve assembly to permit or prevent material flow from the CIC to the delivery unit.

In some embodiments, when associating or detaching the CIC from the delivery unit, at least one valve member is operated automatically, electrically or manually into a ‘close’ configuration.

In some embodiments, the method comprises detaching the delivery unit from the CIC, wherein the delivery unit comprises the cell pellet.

In some embodiments, the method is utilized for isolating enriched cell fractions for use in a method of immune regulation for treating or preventing autoimmune diseases, immune disorders, such as graft versus host disease, COVID-19, acute respiratory distress syndrome, and others; in a method for tissue regeneration, e.g., fat grafting, hair growth, bone regeneration, nerve regeneration, skin rejuvenation, wound healing and others; or in a method for treating or preventing stroke, perianal fistulas, rheumatic disease, general arthritis, osteoarthritis and others.

In some embodiments, the method is for isolating SVF cells from fat.

In some embodiments, the isolating step is an enzymatic or non-enzymatic method step.

In another aspect there is provided a method for separating a cell population from a tissue, the method comprising:
disrupting a tissue in a device comprising a CIC unit and a detachable delivery unit, using a tissue disruption unit; the CIC being associated with a first delivery unit, as disclosed herein;
optionally washing the suspension of cells within the CIC, optional CIC is attached to the delivery unit;
optionally transferring the CIC into a centrifuge and centrifuging to thereby separate cells or a cell pellet, optionally when the CIC is attached to the delivery unit;
permitting the cells or cell pellet of a first fraction to settle into the delivery unit being attached to and in a material communication with said CIC;
disconnecting said first detachable delivery unit containing the cell pellet (following closing one or more of the valve members, as disclosed herein);
treating the disrupted tissue present in the CIC with an enzyme solution to cause digestion of the tissue in the CIC to separate cells or a cell pellet of a second fraction (wherein the cells or pellet of the first and second fractions are of the same or different composition);
optionally washing the suspension of cells within the CIC;
attaching a second delivery unit and permitting the cells or cell pellet of the second fraction to settle into the second delivery unit.

In some embodiments, the method comprises transferring the CIC or the delivery unit into a centrifuge and centrifuging the CIC or unit to thereby isolate a second cell pellet.

In some embodiments, the cells or cell pellet of the second fraction is washed to remove excess enzyme.

In some embodiments, the cellular composition of the first and second fractions is substantially the same.

In some embodiments, the first and second pellets or fractions are combined.

The invention further provides a method for separating a cell population from a tissue, the method comprising:
disrupting or digesting a tissue in a device comprising a CIC unit and a detachable delivery unit, using a tissue disruption unit or an enzyme, respectively, to separate therefrom cells or a pellet of cells; the CIC being associated with a first delivery unit, as disclosed herein;
optionally washing the suspension of cells within the CIC, optionally when the CIC is attached to the delivery unit;
permitting the cells or cell pellet of a first fraction to settle into the delivery unit being attached to and in a material communication with said CIC;
disconnecting said first detachable delivery unit containing the (following closing one or more of the valve members, as disclosed herein);
disrupting or digesting tissue remaining in the CIC using a tissue disruption unit or an enzyme to separate therefrom a further amount of cells or a pellet of cells;
optionally washing the further amount of the cells within the CIC;
attaching a second delivery unit and permitting the further amount of cells or cell pellet settle into the second delivery unit; and
repeating the method one or more additional times to separate a plurality of cell amounts, wherein each amount is provided in a different delivery unit.

In some embodiments, any of the methods disclosed herein further comprises separation of fat or any tissue fraction from the CIC into the delivery unit.

In some embodiments, methods of the invention allow for collecting in one or a plurality of delivery units isolated cell populations, cell pellets, fat, tissue fraction, processing medium or any combination thereof as may be the case.

In some embodiments, once separated into one or more delivery units, the delivery units may be centrifuged to e.g., separate a cell pellet from the medium. In some embodiments, the cell population present in a CIC or the CIC containing processed tissue and cell is not centrifuged. In some embodiments, the delivery unit is provided with an inner membrane enabling separation based on size during centrifugation.

In some embodiments of systems and methods of the invention, delivery units containing cell populations or cell pellets may be stored for long period of time and may thereafter be used as disclosed herein. In order to ensure cell viability during storage, the cells in the delivery unit may be frozen at low temperatures to preserve and protect their attributes. Cryo-preservation may be achievable by treating the cells or sample in the delivery unit with on more agents allowing for cryopreservation. The agents may include one or more cryoprotectants or a freeze medium. Non-limiting examples of cryoprotectants include dimethyl sulfoxide (DMSO), glycerol, alginate, polyvinyl alcohol, and others. Following thawing of the content of the delivery unit, the cryoprotectant may or may not be washed of.

Cryopreservation or cryo-storage at temperatures below −50° C., and at times below −80 or −100° C., requires that a delivery unit of the invention is formed of a material that can withstand low temperatures. Such materials may be polymeric materials, glass and various composite materials, as known in the art. Thus, while generally systems of the invention may be formed of any material, including polymers, glass, composi etc, in cases of cryo-storage, the delivery unit may be formed of such stage materials.

The invention further provides a kit of parts for assembling a device for separating cell population from a tissue, the kit comprising:
a container unit (CIC) for separating cell population from tissues containing same, as disclosed herein;
a detachable delivery unit, as disclosed herein; and
instructions for assembly and/or use.

The invention further provides a kit comprising a CIC according to the invention configured for attachment to a delivery unit according to the invention and instructions for assembling the CIC to the delivery unit and using same.

Further provided is a kit comprising a delivery unit according to the invention configured for attachment to a CIC unit according to the invention and instructions for assembling the CIC to the delivery unit and using same.

In some configurations, the device and method of the invention are adapted for isolating enriched cell populations from an adipose tissue obtained from a subject. The tissue may be any cells and/or tissue fragments that are isolated from a subject. The enriched fraction may be an isolate a stromal cell fraction (SVF), as known in the art.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 depicts a schematic representation of a system of the invention.

FIG. 2 shows a bottom view of a system of the invention.

FIG. 3 presents a side view of a system according to some embodiment of the invention.

FIG. 4 shows a presentation of a delivery unit according to the invention.

FIGS. 5A and B provide exemplary valve and connector members according to some embodiments of the invention.

### DETAILED DESCRIPTION OF EMBODIMENTS

As disclosed herein, the invention concerns a device comprising a cell isolation chamber (or unit) detachably associated with a cell delivery unit, the cell isolation chamber is adapted to receive a tissue to be manipulated to separate therefrom cells or a cell population, wherein the device is configured to allow cells isolated from the tissue in the cell isolation chamber to gravitationally collect in the detachable cell delivery unit.

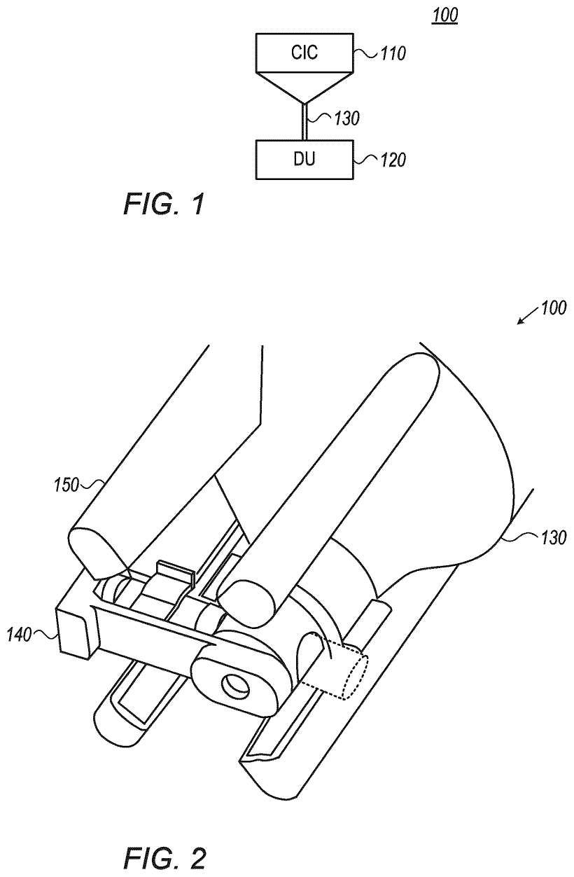

FIG. 1 provides a schematic representation of a system according to the invention. In most general terms, a system 100 comprises a cell isolation chamber CIC or unit 110 and a delivery unit (DU) 120 which are detachably connected through a connector 130 associated to a valve assembly for affecting material communication from said CIC 110 for receiving into the delivery unit 120 an amount of the CIC content.

A bottom view of a system 100 showing the CIC unit 130 and a delivery unit 140, detachably associated, and supporting legs 150 is shown in FIG. 2.

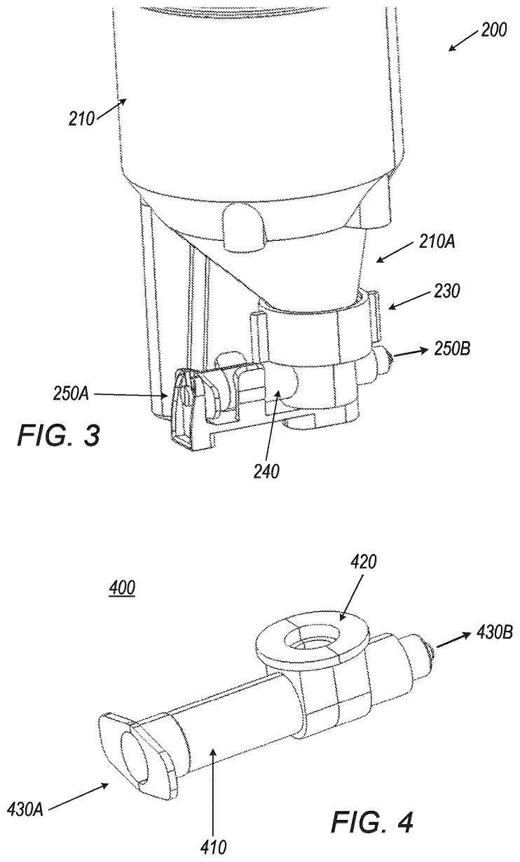

FIG. 3 depicts another embodiment of a system 200 according to the invention. As shown, the system comprises a CIC unit 210 for separating/isolating cells or a cell population from tissues containing same. The container unit 210 having a conical shaped end 210A that is associated to the delivery unit 240 via connector 230. The CIC and delivery unit are in material communication for receiving into the delivery unit 240, separated/isolated cells or cell population or tissues. The detachable delivery unit 240 has a main body (e.g., a barrel) and a reciprocating pump unit 250A for expelling said separated/isolated cells or cell population (or a pellet of same) or tissues via outlet 250B.

An exemplary structure of a delivery unit is provided in FIG. 4. As shown, the unit 400 has a main body (e.g., a barrel) 410 and a reciprocating pump unit 430A for expelling separated/isolated cells or cell population (or a pellet of same) or tissues therefrom through outlet 430B in a direction shown by the arrow. The unit may be used as a syringe for systemic delivery of the cells, e.g., intravenously or intra-arterial, intramuscular or intraperitoneal delivery; or locally by intra-articular, into the spine, into the scalp (hair growth), into the face/breast with or without a fat graft, in a scaffold (bone regeneration, breast degradable scaffolds) and by other means.

The main body 410 is shaped as a barrel having at least two openings: one positioned opposite the plunger path through which cells may be expelled (outlet 430B), and the other 420 positioned along the barrel and permitting liquid communication with said main unit.

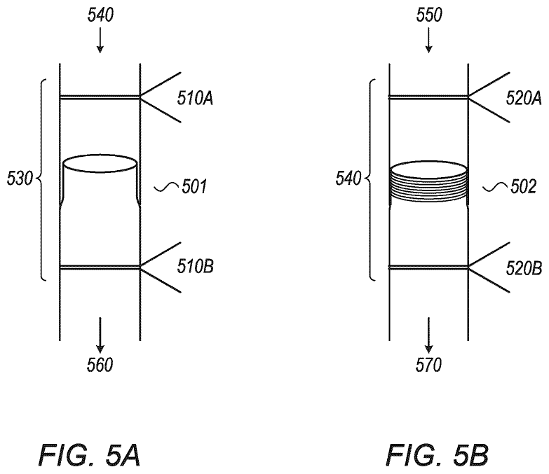

As further shown in FIG. 3, delivery unit 240 may be connected with of the conical CIC 210 via a connector member or fittings 230. To securely assemble the CIC 210 and the delivery unit 240, each is provided with a connector member 501 or 502 which may be a male or female member having a threaded or non-threaded end(s); a male or female barb connector; a coupling adaptor member; a double tapped bushing adaptor; and others, as exemplified in FIG. 5A and FIG. 5B. The one or both connector members may be associated to a valve member 510A and 510 B or 520A and 520B to form a valve assembly 530 and 540, respectively, such that once both the CIC and the delivery unit are securely assembled via a connector member 501 or 502 provided on each respectively, a valve assembly may be operated to controllably permit liquid communication from the CIC to the delivery unit. The valve assembly 530 or 540 may comprise one or more valve members 510A and 510B; and 520A and 520B, respectively, which together are designed to ensure liquid communication from the CIC 540 or 550 to the delivery unit 560 or 570, respectively, upon demand and in a controllable manner.

Each of the valve members 510A, 510B, 520A 520B may be a one way-flap member that allows flow in a direction from the CIC 540 or 550 to the delivery unit 560 or 570 and stops flow in the direction of origin.

In some embodiments, a valve member 510A, 510B, 520A 520B may be an electric valve operated by a motor or a mechanical valve member operated manually.

In some embodiments, one or more of the valve members 510A, 510B, 520A 520B is a manual valve. Manual valve members may be in a form selected from levers, pedals, hand knobs or wheels to crank a screw-turned stem.

In some embodiments, the valve assembly 530 or 540 is or comprises a single valve member that is provided in the connector member of the CIC unit or in the connector member of the delivery unit.

In some embodiments, the valve assembly 530 or 540 is or comprises a double valve having two valve members, one associated with the CIC unit and another with the delivery unit.

In a double valve unit such as that depicted in FIGS. 5A and B, the two valves 510A and 510 B or 520A and 520B define an upper valve body (associated with the CIC 540 or 550) and a lower valve body (associated with the delivery unit 560 or 570), which may have the same structure, and may be coaxially formed. The two ends of the upper valve body and the two ends of the lower valve body are integrally conne respective units and to the two connector members 501 or 502.

The device of the invention may be assembled such that the CIC unit is above the delivery unit to enable gravitational collection of the CIC content without external intervention. The CIC may thus be equipped with a base or a stage or a leg assembly as shown in FIG. 2, which maintains the CIC at a distance from the surface on which it is positioned sufficient to fit the delivery unit at its lower part. Thus, in some embodiments, the body of the device or CIC unit is supported on legs which may be adjustable to a distance convenient for operation.

1-44. (canceled)

45. A device comprising a cell isolation chamber (CIC) detachably associated with a cell delivery unit, the CIC being adapted to receive a tissue to be mechanically and/or enzymatically manipulated to separate therefrom cells or cell populations, wherein the device is configured to allow cells isolated from the tissue in the cell isolation chamber to gravitationally collect in the detachable cell delivery unit, wherein the delivery unit is configured for delivery of the isolated cells in vivo or in vitro.

46. The device according to claim 45, the device comprising:
a cell isolation chamber (CIC) configured for separating/isolating cells or a cell population from tissues containing same, said CIC being adapted to hold the tissue and a medium optionally containing an enzyme;
optionally a tissue disruption element for mixing or mechanically disrupting the tissue to obtain the cells or the cell population;
a delivery unit being detachably connected to the CIC through a connector associated to a valve assembly for receiving thereinto an amount of the CIC content;
wherein the delivery unit having a main body and a reciprocating member configured for expelling its content.

47. The device according to claim 45, wherein the CIC is provided with an element or a member configured for inducing vortexing, shaking or stirring, wherein the member is not a blade or a set of blades.

48. The device according to claim 45, wherein the delivery unit is shaped and operated as a storage unit or as a syringe for delivery of cells.

49. The device according to claim 45, wherein the delivery unit has a main body shaped as a barrel or an elongated tubular structure comprising a reciprocating unit or a plunger and a plunger rod that is configured for linear movement within the barrel.

50. The device according to claim 49, wherein the barrel has at least two openings, one of said opening being positioned along a path of material communication with said CIC and another at one end of the barrel being opposite to the plunger path.

51. The device according to claim 45, wherein the delivery unit is provided with a membrane or a filter disposed perpendicularly to a main axis of a tubular structure of the unit.

52. The device according to claim 45, wherein the delivery unit is connected with the CIC through a connector member.

53. The device according to claim 52, wherein the connector member is a male or female member having a threaded or non-threaded end(s); a male or female barb connector; a coupling adaptor member; or a double tapped bushing adaptor.

54. A method for isolating a cell population from a tissue, the method comprising:
disrupting or digesting the tissue in a device comprising a cell isolation chamber (CIC) and a delivery unit detachably associated with the CIC, using a tissue disruption unit or an enzymatic solution present in CIC to obtain a cell suspension;
optionally washing the suspension of cells within the CIC, optionally when the container unit is attached to the delivery unit; and
permitting the cells or cell pellet to settle into the delivery unit through a valve assembly.

55. The method according to claim 54, wherein the tissue is treated when the delivery unit is detached from the CIC; wherein prior to permitting the cells or cell pellet to settle into the delivery unit, the delivery unit is attached to the CIC and the valve assembly is positioned to provide material flow into the delivery unit.

56. The method according to claim 54, wherein the cells or cell pellet are/is gravitationally collected in the delivery unit.

57. The method according to claim 54, wherein the cells or cell population are collected in one delivery unit which is detached and replaced with another delivery unit to receive another fraction of the cells or to receive another portion of the CIC content.

58. The method according to claim 57, wherein when associating or detaching the CIC from the delivery unit, at least one valve member is operated automatically, electrically or manually into the ‘close’ configuration.

59. The method according to claim 54, wherein the method comprises detaching the delivery unit from the CIC.

60. The method according to claim 54, for isolating enriched cell fractions for use in a method of immune regulation for treating or preventing autoimmune diseases, immune disorders, such as graft versus host disease, COVID-19, acute respiratory distress syndrome; in a method for tissue regeneration; or in a method for treating or preventing stroke, perianal fistulas, rheumatic disease, general arthritis, osteoarthritis; or, for isolating SVF cells from fat.

61. The method for separating a cell population from a tissue according to claim 54, the method comprising:
disrupting a tissue in a device comprising a CIC unit and a detachable delivery unit, using a tissue disruption unit; the CIC being associated with a first delivery unit;
optionally washing the suspension of cells within the CIC, optionally when the CIC is attached to the delivery unit;
optionally transferring the CIC into a centrifuge and centrifuging to thereby separate cells or a cell pellet, optionally when the CIC is attached to the delivery unit;
permitting cells or cell pellet of a first fraction to settle into the delivery unit being attached to and in a material communication with said CIC;
disconnecting said first detachable delivery unit;
treating the disrupted tissue present in the CIC with an enzyme solution to cause digestion of the tissue in the CIC to separate cells or a cell pellet of a second fraction;
optionally washing the suspension of cells within the CIC;
attaching a second delivery unit and permitting the cells or cell pellet of the second fraction to settle into the second delivery unit.

62. The method for separating a cell population from a tissue according to claim 54, the method comprising:
disrupting or digesting a tissue in a device comprising a CIC unit and a detachable delivery unit, using a tissue disruption unit or an enzyme, respectively, to separate therefrom cells or a pellet of cells; the CIC being associated with a first delivery unit;
optionally washing the suspension of cells within the CIC, optionally when the CIC is attached to the delivery unit;
permitting the cells or cell pellet of a first fraction to settle into the delivery unit being attached to and in a material communication with said CIC;
disconnecting said first detachable delivery unit containing the cell pellet;
disrupting or digesting tissue remaining in the CIC using a tissue disruption unit or an enzyme to separate therefrom a further amount of cells or a pellet of cells;
optionally washing the further amount of the cells within the CIC;
attaching a second delivery unit and permitting the further amount of cells or cell pellet settle into the second delivery unit; and
repeating the method one or more additional times to separate a plurality of cell amounts, wherein each amount is provided in a different delivery unit.

63. The method according to claim 62, for collecting in one or a plurality of delivery units isolated cell populations, cell pellets, fat, tissue fraction, processing medium or any combination thereof.

64. A method for isolating different fractions of a cell population from a tissue, the method comprising:
disrupting or digesting the tissue in a device comprising a cell isolation chamber (CIC) and a delivery unit detachably associated with the CIC, using a tissue disruption unit or an enzymatic solution present in CIC to obtain a cell suspension; and
permitting the suspension or a part of the CIC content to settle into the delivery unit through a valve assembly,
wherein the disrupting or digesting is repeated one or more times on the tissue contained in the CIC to further separate additional fractions of cells into different delivery units.
